(19) United States
(12) Patent Application Publication
(10) Pub. No.: US 2006/0129059 A1
(43) Pub. Date: Jun. 15, 2006

(54) Attitude correction apparatus and method

(75) Inventors: Soo-kwan Kim, Seongnam-si (KR); Jin-sang Hwang, Suwon-si (KR); Kyung-ho Kim, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/291,961

(22) Filed: Dec. 2, 2005

(30) Foreign Application Priority Data
Dec. 11, 2004 (KR) 10-2004-0104621

(51) Int. Cl. A61B 5/04; A61B 5/103

(52) U.S. Cl. 600/546; 600/595

(57) ABSTRACT

An attitude correction and method are provided. In the attitude correction apparatus, an electromyogram measuring unit is attached to a subject to obtain an electromyogram waveform and calculate muscle tension from the electromyogram waveform. An attitude measuring unit is attached to the subject to measure a subject's attitude. A controlling unit outputs a warning signal when the subject's muscle tension and attitude are out of a predetermined range.

### CROSS-REFERENCE TO RELATED PATENT APPLICATION

This application claims priority from Korean Patent Application No. 10-2004-0104621, filed on Dec. 11, 2004, in the Korean Intellectual Property Office, the disclosure of which is incorporated herein in its entirety by reference.

### BACKGROUND OF THE INVENTION

1. Field of the Invention

Apparatuses and methods consistent with the present invention relate to attitude correction, and more particularly, to attitude correction including testing a subject's electromyogram and checking a subject's attitude using a position sensor.

2. Description of the Related Art

The joints of the human body behave differently and maintain a stable state, depending on the action of the adjacent muscles. When a single attitude or position is maintained for a long time, or when a lifestyle or working environment is bad, muscular function may deteriorate and soft tissue adjacent to the joints may harden. As a result, the joints may become unbalanced and a patient's attitude may change. Such an attitude change may lead to musculoskeletal pain caused by biostress.

Patients who suffer from chronic pain often show an abnormal change in attitude. The attitude change may be caused by various factors, such as patient's genetic inclination, psychological behavior, lifestyle, and working environment. If the attitude change is prolonged, forces acting on each joint and muscle become unbalanced. These unbalanced forces may cause various musculoskeletal pains. When a patient complains of arthritis pain, arthritis therapy is important; however, interest in the patient's attitude change, due to the abnormal stress on the joints, should be taken. If the patient's attitude change can be corrected, the arthritis and its pain may be eliminated more quickly than with arthritis therapy alone. Additionally, future reoccurrences of the arthritis can be prevented.

Attitude analysis involves an evaluation of the patient's muscles and attitude. This attitude analysis makes it possible to determine the relationships between muscle tension and body arrangement.

### SUMMARY OF THE INVENTION

The present invention provides an attitude correction apparatus and method for correcting a subject's attitude based on a measured electromyogram of the subject.

According to an aspect of the present invention, there is provided an attitude correction apparatus, including: an electromyogram measuring unit attached to a first portion of a subject to obtain an electromyogram waveform at the first portion and calculate muscle tension from the electromyogram waveform; an attitude measuring unit attached to a second portion of the subject to measure a subject's attitude at the second portion; and a controlling unit outputting a warning signal when the subject's muscle tension and attitude are out of a predetermined range.

According to another aspect of the present invention, there is provided an attitude correction method, including: obtaining an electromyogram waveform at a predetermined portion of a subject and calculating a muscle tension from the electromyogram waveform; when the muscle tension is greater than or equal to a predetermined value, detecting a change of a subject's attitude for a predetermined time and determining whether the change of the subject's attitude is within an attitude change range; and when the change of the subject's attitude is within the attitude change range, outputting a warning to require the subject to change the attitude.

### BRIEF DESCRIPTION OF THE DRAWINGS

The above and other aspects of the present invention will become more apparent by the following detailed description of exemplary embodiments thereof with reference to the attached drawings in which:

FIG. 1 illustrates an attitude correction apparatus attached to a subject according to an exemplary embodiment of the present invention;

FIG. 2 is a block diagram of the attitude correction apparatus shown in FIG. 1; and

FIG. 3 is a flowchart illustrating an attitude correction method according to an exemplary embodiment of the present invention.

### DETAILED DESCRIPTION OF EXEMPLARY EMBODIMENTS OF THE INVENTION

The present invention will now be described more fully with reference to the accompanying drawings, in which exemplary embodiments of the invention are shown.

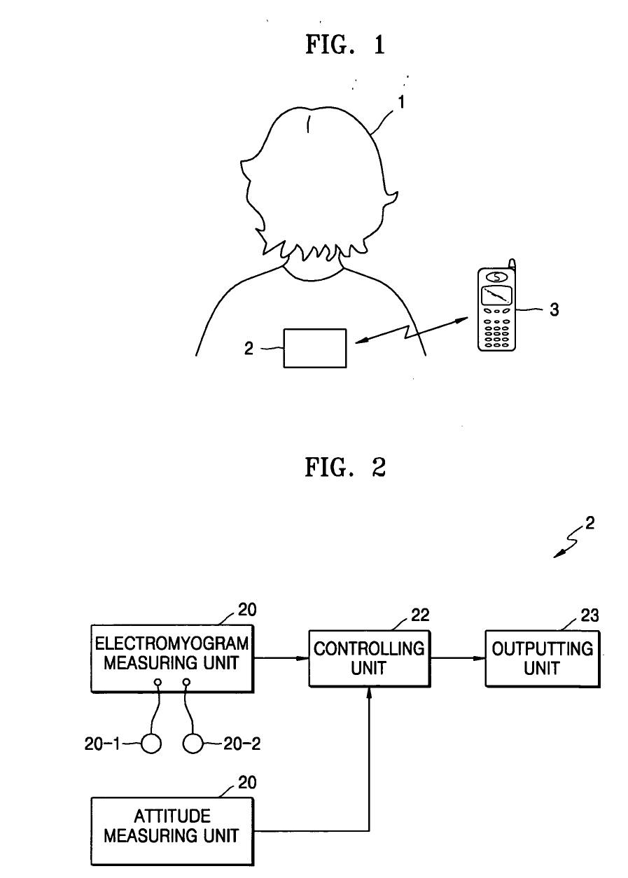

FIG. 1 illustrates an attitude correction apparatus 2 attached to a human subject 1 according to an exemplary embodiment of the present invention.

The attitude correction apparatus 2 may be attached to a subject's shoulder, back, or waist, or to another part of the body as would be understood in the art, and measures a subject's electromyogram over a predetermined period. By measuring muscle tension, the attitude correction apparatus 2 determines when the subject 1 maintains a single attitude for a long period of time or when the subject assumes an improper attitude, and gives an alarm or a warning message which may be provided through the mobile terminal 3 to the subject 1 indicating that a change in attitude should be made. In this manner, it is possible to instruct the subject 1 to maintain a proper attitude.

The mobile terminal 3 may be a separate portable device, a hand-held phone, a personal digital assistant (PDA), a notebook computer, or another device, as would be understood by one of skill in the art, which can provide a display function or an audio output. The mobile terminal 3 carried by the subject 1 may receive and process a warning signal transmitted from the attitude correction apparatus 2 and may display the processed signal or may output an audio signal.

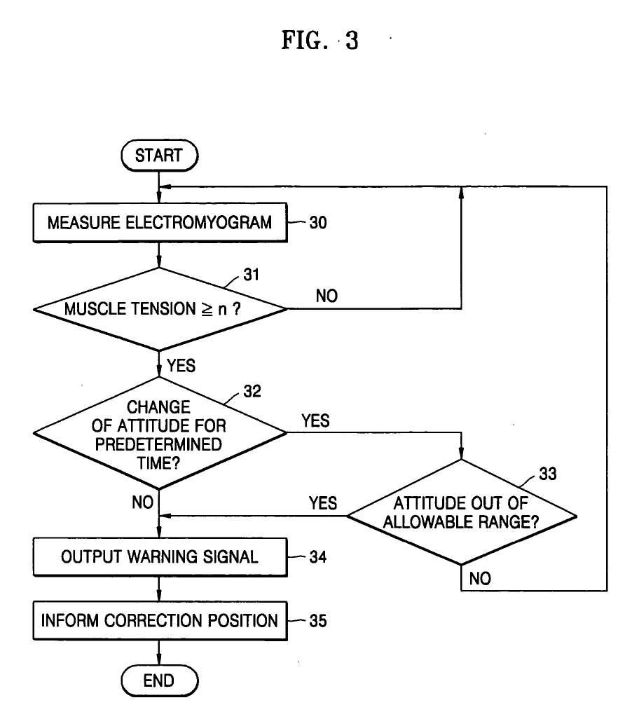

FIG. 2 is a block diagram of the attitude correction apparatus shown in FIG. 1. Referring to FIG. 2, the attitude correction apparatus 2 includes an electromyogram measuring unit 20 having at least two measuring terminals 20-1 and 20-2, an attitude measuring unit 21, a controlling unit 22, and an outputting unit 23.

FIG. 3 is a flowchart illustrating an attitude correction method according to an exemplary embodiment of the present invention. An operation of the attitude correction apparatus shown will now be described with reference to FIGS. 1 through 3.

In operation 30, the electromyogram measuring unit 20 periodically measures a muscle tension through two measuring terminals 20-1 and 20-2. Electromyography (EMG) is a process of graphically recording electrical activity of a muscle during the contraction of a skeletal muscle. Through the EMG, an abnormality in the muscle can be determined by measuring the electrical activity of the skeletal muscle. There are two types of the EMG: needle EMG (nEMG) and surface EMG (sEMG). nEMG uses a needle which is inserted and guided within a muscle tissue to measure muscle action potential during a stable state or an active state. Meanwhile, sEMG uses electrodes attached on the skin. Thus, sEMG is non-invasive and painless.

An electromyogram can diagnose a functional abnormality of muscle, though not a structural characteristic thereof. It is known that sEMG has a high clinical value in diagnosing a Repetitive Strain Injury (RSI) caused by a repetitive use of muscle, a Work Related Musculoskeletal Disorder (WRMD) caused by excessive work, a Myofascial Pain Syndrome (MPS), causing a pain in a specific muscle portion called a Trigger Point, and a Chronic Fatigue and Immune Dysfunction Syndrome (CFIDS).

Although sEMG is described below, the present invention is not limited to sEMG.

One of the two measuring terminals 20-1 and 20-2 is a reference terminal and the other is an output terminal which outputs a Motor Unit Action Potential Trains (MUAPT) waveform based on a relative potential difference with respect to the reference terminal. The electromyogram measuring unit 20 determines muscle tension by calculating analysis parameters, including a root mean square (RMS), a median power frequency, a mean frequency, and an RMS cross-correlation from the MUAPT waveform.

The RMS reflects muscle contraction or muscle tension, and the median power frequency or intermediate frequency reflects muscle fatigue. Also, the cross-correlation between muscle regions reflects muscle contraction timing. The controller 22 calculates the muscle tension using the above-described quantitative analysis.

The attitude measuring unit 21 measures the subject's attitude. The attitude measuring unit 21 may be a tilt sensor which measures a tilt with respect to at least two axes, including a horizontal axis and a vertical axis, or may be a two-axis acceleration sensor which measures an acceleration with respect to a motion of the subject. In the case of the two-axis acceleration sensor, the subject's attitude can be measured by calculating a pitch angle and a roll angle with respect to a horizontal axis and a vertical axis, respectively, using a DC component of the measured acceleration signal. Consequently, the attitude measuring unit 21 outputs tilt angles of the subject with respect to the horizontal axis and the vertical axis.

According to the present invention, it is assumed that the proper or correct attitude means that a first straight line, connecting the shoulders of the subject, is parallel to the ground and that a second straight line, connecting the head and backbone of the subject, is perpendicular to the ground. When the attitude measuring unit 21 is attached to a center portion of the subject's back, between the shoulders or is attached to a mid-waist region, values of the correct attitude in the attitude measuring unit 21 are set so that the horizontal axis and the vertical axis of the attitude measuring unit 21 are respectively parallel to the first straight line and the second straight line, respectively. As one example, when the attitude measuring unit 21 is attached to a center portion of the subject's back, between the shoulders, its horizontal axis is set to be parallel to the first straight line and its vertical axis is set to be parallel to the second straight line. As another example, when the attitude measuring unit 21 is attached to one shoulder, the horizontal axis and the vertical axis of the measuring unit 21 are adjusted in accordance with the natural tilt of the subject's shoulder when the subject's back is maintained in the correct attitude so that the horizontal axis is set as tilted as the shoulder does from the subject's correct attitude and its vertical axis is set to perpendicular to the set horizontal axis in the subject's normal condition.

If the muscle tension is determined to be greater than or equal to the predetermined value n, in operation 31, then the process moves to operation 32, where it is determined whether there has been a change of attitude, as measured by the attitude measuring unit 21, within a predetermined period of time. The predetermined period of time can be determined experimentally, and may be, for example, 30 minutes. If it is determined that the attitude has not changed during the predetermined period of time, the controlling unit 22 outputs a warning signal in operation 34.

If it is determined, in operation 32, that the attitude has changed during the predetermined period of time, then the process moves to operation 33, where it is determined whether the new attitude is outside an allowable range. The allowable range is a range of tilt in which muscle tension is not caused. This range may be obtained experimentally. If the attitude is outside the allowable range, the controlling unit 22 outputs a warning signal in operation 34.

Thereby, according to operations 31, 32 and 34, if the muscle tension value output from the electromyogram measuring unit 20 is greater than a predetermined value and there is not a change in attitude for a predetermined time period, for example 30 minutes, as measured by the attitude measuring unit 21, the controlling unit 22 gives a warning instructing the subject to change his or her current attitude.

Further, according to operations 31, 32, 33 and 34, if it is determined that the muscle tension is greater than the predetermined value n, there is an attitude change for the predetermined time period, and the subject's new attitude is outside the allowable range, the controlling unit 22 gives the warning to the subject. If the muscle tension is greater than the predetermined value n and there is an attitude change for the predetermined time period, and the subject's new attitude is within the allowable range, this means that only a short time has passed since the subject adjusted his/her attitude to be the current attitude. Therefore, the process returns to operation 30 of periodically measuring the electromyogram.

The warning is output to the subject from the outputting unit 23. In operation 35, the controlling unit 22 can give a simple warning through the outputting unit 23 or can provide an attitude correction instruction to the mobile terminal 3. For example, the attitude correction instruction may be information on how much the subject should correct his or her attitude with respect to the horizontal or vertical axis.

The outputting unit 23 can support a local area communication method, such as Bluetooth communication, so as to communicate with the mobile terminal 3. The mobile terminal 3 can display the warning or the attitude correction instruction output from the controlling unit 22, or can output the warning or instruction in the form of an audio signal.

According to the present invention, pain caused by an incorrect attitude can be lessened by correcting the subject's attitude and balancing the muscular strength applied to the joints.

The present invention can also be embodied in computer-readable codes on a computer-readable recording medium. The computer-readable recording medium is any data storage device that can store data which can be read, thereafter, by a computer system. Examples of a computer-readable recording medium include read-only memory (ROM), random-access memory (RAM), CD-ROMs, magnetic tapes, floppy disks, optical data storage devices, and carrier waves (such as data transmission through the Internet). The computer-readable recording medium can also be distributed over network coupled computer systems so that the computer-readable code is stored and executed in a distributed fashion. Also, functional programs, codes, and code segments for accomplishing the present invention can be easily construed by programmers skilled in the art to which the present invention pertains.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An attitude correction apparatus comprising:
an electromyogram measuring unit which obtains an electromyogram waveform of a subject and calculates a muscle tension of the subject based on the electromyogram waveform;
an attitude measuring unit, which measures an attitude of the subject; and
a controlling unit which outputs a warning signal based on the muscle tension and the attitude.

2. The attitude correction apparatus of claim 1, wherein the attitude measuring unit comprises:
a plurality of tilt sensors which measure tilt with respect to horizontal and vertical axes, or
a two-axis acceleration sensor which measures an acceleration of the subject.

3. The attitude correction apparatus of claim 2, wherein the controlling unit outputs the warning signal if the muscle tension is greater than or equal to a predetermined value and there is not a change of the tilt measured by the attitude measuring unit during a predetermined period of time.

4. The attitude correction apparatus of claim 2, wherein the controlling unit outputs the warning signal if the muscle tension is greater than or equal to a predetermined value, there is a change of the tilt is during a predetermined period of time, and the attitude is outside of an allowable range.

5. The attitude correction apparatus of claim 2, wherein the horizontal axis and the vertical axis are initially set such that the attitude of the subject is not inclined with respect to the vertical axis or the horizontal axis if the subject is in a position in which a first straight line, connecting shoulders of the subject, is parallel to a ground surface and in which a second straight line, connecting a head and a backbone of the subject, is perpendicular to the first straight line.

6. The attitude correction apparatus of claim 5, wherein the controlling unit outputs the warning signal if the muscle tension is greater than or equal to the predetermined value and there is not a change of the tilt measured by the attitude measuring unit during a predetermined period of time.

7. The attitude correction apparatus of claim 6, wherein the controlling unit outputs the warning signal if the muscle tension is greater than or equal to the predetermined value, there is a change of the tilt during a predetermined period of time, and the attitude is outside of an allowable range.

8. The attitude correction apparatus of claim 1, further comprising a mobile terminal which receives the warning signal output by the controlling unit and which outputs an audible warning signal or displays an attitude correction instruction.

9. An attitude correction method comprising:
obtaining an electromyogram waveform of a subject and calculating a muscle tension from the electromyogram waveform;
if the muscle tension is greater than or equal to a predetermined value, detecting an attitude of the subject and determining whether there is change of the attitude during a predetermined period of time; and
if there is a change of the subject's attitude and the attitude of the subject is not within the attitude change range, outputting a warning signal.

10. The attitude correction method of claim 9, further comprising:
if the muscle tension is greater than or equal to the predetermined value and there is a change of the attitude during the predetermined time, determining whether the attitude is within an allowable range; and
if the attitude is outside of the allowable range, outputting a warning.

11. The attitude correction method of claim 9, wherein the warning is output through a mobile terminal.

12. The attitude correction method of claim 11, wherein the mobile terminal provides attitude correction information to the subject after outputting the warning.

13. The attitude correction apparatus of claim 1, wherein the measuring unit is attached to a first portion of a subject, and the attitude measuring unit is attached to a second portion of the subject which is different from the first portion.
